(19) United States
(12) Patent Application Publication
(10) Pub. No.: US 2024/0189061 A1
(43) Pub. Date: Jun. 13, 2024

(54) ULTRASOUND GENERATOR FOR INHIBITING INTESTINAL INFLAMMATORY FACTOR AND/OR IMPROVING NEUROINFLAMMATION AND ITS SYSTEM AND METHOD THEREOF

(71) Applicant: National Yang Ming Chiao Tung University, Taipei (TW)

(72) Inventor: Feng-Yi YANG, Taipei City (TW)

(21) Appl. No.: 18/194,743

(22) Filed: Apr. 3, 2023

(30) Foreign Application Priority Data
Dec. 13, 2022 (TW) 111147845

(51) Int. Cl. A61B 90/00; A61N 7/00

(52) U.S. Cl. CPC A61B 90/36; A61N 7/00

(57) ABSTRACT

The system includes: an ultrasound imaging probe located in the center of the ultrasound probe device; an ultrasound stimulation probe located around the center of the ultrasound probe device, and two independent piezoelectric elements with a slight frequency difference can focused ultrasound and applied to the same target area, and fixed on a specific position of the abdomen with a touch fastener to generate an acoustic field that changes the intensity of the acoustic field with time: Stimulate the area of the inflammatory bowel in the abdomen through this acoustic field: to suppress the inflammatory bowel disease and the brain inflammation through the gut-brain axis.

### CROSS-REFERENCE TO RELATED APPLICATIONS

This non-provisional application claims priority under 35 U.S.C. § 119 on Patent Application No. TW111147845 filed in Taiwan, Republic of China Dec. 13, 2022, the entire contents of which are hereby incorporated by reference in its entirety.

### FIELD OF THE INVENTION

The present invention relates generally to a type of ultrasound which can be applied to abdomen, it is applicable to regulating immune and nervous responses, alleviating, recovering or preventing neurodegenerative disease resulted from brain inflammation induced by chronic inflammation, and more particularly to an ultrasound generator and system which uses ultrasound field to inhibit intestinal inflammatory factor and/or to improve neuroinflammation.

### BACKGROUND OF INVENTION

Inflammatory intestinal tract disease is a chronic inflammation disease of gastrointestinal tract. Clinical manifestations include diarrhea, abdominal pain, gastrointestinal bleeding and loss of weight. The present therapeutic methods include antibiotics, steroid, immunosuppression, neutralizing antibody and operation, which have limited therapeutic effect, only alleviate symptoms. This kind of diseases will induce cardiovascular disease, metabolic syndrome, nervous and mental diseases to patients. Long-term chronic inflammation may induce cancer. At present, a therapeutic method for alleviation, recovery or prevention and free from long-term medication is required.

Using novel lactobacilli to adjust intestinal microflora results in immunomodulation and anti-inflammatory effect, as well as enhances the cognitive function of brain, improves affective disorder and treats or prevents neurodegenerative disease. These mechanisms mediate the relationship between brain and intestinal tract. The influence path includes vagus, immune system and bacterial metabolite. The product enters blood, and these pathway imbalances will influence the inflammation of cranial nerves or change the permeability of blood-brain barrier. Therefore, the neuromodulation technique through gut-brain axis is a new technique to be developed urgently.

The present invention provides an abdominal ultrasound device, which uses abdominal ultrasound stimulation to regulate intestinal flora, inhibit microglia activation through gut-brain axis and increase BDNF (brain-derived neurotrophic factor) expression level, so as to improve relevant neurodegenerative disease induced by brain inflammation.

### SUMMARY OF THE INVENTION

In view of this, the present invention discloses an ultrasound generator and system which uses ultrasound field to inhibit intestinal inflammatory factor and/or to improve neuroinflammation. The ultrasound probe of the device is designed as an integrated single probe with a diagnostic probe in the center and a focusing probe in peripheral region. The system is designed as a hands-free fixed abdominal ultrasound stimulation system. The abdominal ultrasound probe is fixed to a specific position of abdomen by girdle and Velcro for irradiation. The irradiated position can be adjusted elastically by the girdle, or one probe or multiple probes are fixed to a specific position or different positions for synchronous irradiation.

The present invention used 0.5 W/cm² and 1.0 W/cm² Low-intensity pulsed ultrasound (LIPUS) respectively to stimulate the mouse abdomen with Lipopolysaccharide (LPS) induced systemic inflammation under specific parameters. The direct effect of abdominal ultrasound stimulation on the colon and the indirect effect on the brain were evaluated according to the anti-inflammatory effect.

FIGS. 5a and 5b show the 7 days' experiment data of mice. Intraperitoneal injection of 0.75 mg/kg LPS is applied to the laboratory mice every day during seven consecutive days to induce systemic inflammation. On Day 2 of experiment, the mice with LPS-induced systemic inflammation received LIPUS treatment every day during 6 consecutive days. On the last day of experiment, the mice's colons and cerebral cortices were taken out, and the western blotting was used for protein analysis.

Analysis result: it is found in the abdomen of the mouse with LPS-induced systemic inflammation stimulated by 0.5 W/cm² ultrasound that the overactivation of astrocytes in cerebral cortex is inhibited indirectly, and the expression level of inflammatory factor (IL-6 [symbol] IL-1 [symbol] COX-2) and apoptosis factor (Cleaved Caspase-3) is inhibited by mitigating the pro-inflammatory reaction biological pathway mediated by TLR4/NF-κB. It is found in the abdomen of the mouse with LPS-induced systemic inflammation stimulated by 1.0 W/cm² LIPUS that the colonic inflammatory factor and apoptosis factor and the expression level of tight junction protein (Occludin [symbol] ZO-1) can be inhibited effectively, and the tight junction protein of cerebral cortex can be improved.

FIG. 6 shows the expression level of inflammatory factor and apoptosis factor in the colon tissue of laboratory mouse resulted from ultrasound stimulation in abdomen. This system uses ultrasound to stimulate the abdomen, and the expression level of inflammatory factor and apoptosis factor in the colon tissue of laboratory mouse is obtained. When an organism receives the stimulation of LPS, the inflammatory factor and apoptosis factor are generated by immunoreaction, causing the inflammation of intestinal tract. When the LIPUS treatment is finished, the changes in the manifestation of inflammatory factor and apoptosis in the colon tissue can be observed by Western blotting. The quantitative statistical result of the content variation of IL-6 in the colon tissue is given below.

A in FIG. 6 shows the IL-6 protein expression level. In comparison to control group (sham), LPS group has significant increase. After LIPUS treatment, in comparison to LPS group, LPS+1.0 W/cm² LIPUS group has a significant inhibitory effect. In comparison to LPS+0.5 W/cm² LIPUS group, LPS+1.0 W/cm² LIPUS group has a significant inhibitory effect.

B in FIG. 6 shows the IL-1β protein expression level. In comparison to sham group, LPS group has significant increase. After LIPUS treatment, in comparison to LPS group, LPS+1.0 W/cm² LIPUS group has a significant inhibitory effect. In comparison to LPS+0.5 W/cm² LIPUS group, LPS+1.0 W/cm² LIPUS group has a significant inhibitory effect.

C in FIG. 6 shows the COX-2 protein expression level. In comparison to sham group, LPS group has significant increase. After LIPUS treatment, in comparison to LPS group, LPS+1.0 W/cm² LIPUS group has a significant inhibitory effect. In comparison to LPS+0.5 W/cm² LIPUS group, LPS+1.0 W/cm² LIPUS group has a significant inhibitory effect.

D in FIG. 6 shows the Cleaved Caspase-3 protein expression level. In comparison to sham group, LPS group has significant increase. After LIPUS treatment, LPS+0.5 W/cm² LIPUS group and LPS+1.0 W/cm² LIPUS group have a significant inhibitory effect in comparison to LPS group (average value±SEM; * represents comparison with the value of sham group, *P<0.05, **P<0.01, ***P<0.001; # represents comparison with the value of LPS group, #P<0.05, ##P<0.01, ###P<0.001; † represents comparison with the value of 0.5 W/cm² LIPUS group, †P<0.05; ††P<0.01; n=5).

The above results show the changes in IL-6, IL-1β, COX-2, and Cleaved Caspase-3 in the colon tissue after LIPUS treatment for the animal model of LPS-induced systemic inflammation. The LIPUS intensity 1.0 W/cm² has a significant effect on inhibiting inflammation and resisting apoptosis.

FIG. 7 shows the expression level of inflammatory factor and apoptosis factor in the cerebral cortex tissue of laboratory mouse resulted from ultrasound stimulation in abdomen. This system uses ultrasound to stimulate the abdomen, and the expression level of inflammatory factor and apoptosis factor in the cerebral cortex tissue of laboratory mouse is obtained. When an organism receives the stimulation of LPS, the inflammatory factor and apoptosis factor are generated by immunoreaction. When the LIPUS treatment is finished, the changes in the manifestation of inflammatory factor and apoptosis in the cerebral cortex tissue can be observed by Western blotting. The quantitative statistical result of the content variation of IL-6 in the cerebral cortex tissue is given below.

A in FIG. 7 show IL-6. In comparison to sham group, LPS group has significant increase. After LIPUS treatment, LPS+0.5 W/cm² LIPUS group has a significant inhibitory effect in comparison to LPS group.

B in FIG. 7 shows IL-1β. In comparison to sham group, LPS group has significant increase. After LIPUS treatment, LPS+0.5 W/cm² LIPUS group and LPS+1.0 W/cm² LIPUS group have a significant inhibitory effect in comparison to LPS group.

C in FIG. 7 shows COX-2. In comparison to sham group, LPS group has significant increase. After LIPUS treatment, LPS+0.5 W/cm² LIPUS group and LPS+1.0 W/cm² LIPUS group have a significant inhibitory effect in comparison to LPS group.

D in FIG. 7 shows the Cleaved Caspase-3. In comparison to sham group, LPS group has significant increase. After LIPUS treatment, LPS+0.5 W/cm² LIPUS group and LPS+1.0 W/cm² LIPUS group have a significant inhibitory effect in comparison to LPS group (average value±SEM; * represents comparison with the value of sham group, *P<0.05, **P<0.01, ***P<0.001; # represents comparison with the value of LPS group, #P<0.05, ##P<0.01, ###P<0.001; n=4).

The above results show the changes in IL-6, IL-1β, COX-2 and Cleaved Caspase-3 in the cerebral cortex tissue after LIPUS abdomen treatment of the animal model of LPS-induced systemic inflammation. The LIPUS intensity 0.5 W/cm² has a significant effect on inhibiting inflammation and resisting apoptosis.

FIG. 8 shows the expression level of TLR4 receiver and NF-κB transcription factor in the cerebral cortex tissue of laboratory mouse resulted from ultrasound stimulation in abdomen. This system uses ultrasound to stimulate the abdomen. The expression level of TLR4 receiver and NF-κB transcription factor in the cerebral cortex tissue of laboratory mouse is obtained. When an organism receives the stimulation of LPS, it is combined with the receiver TLR4, the transcription factor NF-κB transcribes inflammatory factor to make the tissue generate inflammatory reaction. When the LIPUS treatment is finished, the changes in the manifestation of TLR4 and NF-κB in the cerebral cortex tissue can be observed by Western blotting. The quantitative statistical result of the content variation of TLR4 in the cerebral cortex tissue is given below.

A in FIG. 8 shows TLR4 receiver. In comparison to sham group, LPS group has significant increase. After LIPUS treatment, LPS+0.5 W/cm² LIPUS group has a significant inhibitory effect in comparison to LPS group.

B in FIG. 8 shows the NF-κB transcription factor. In comparison to sham group, LPS group has a significant decrease. After LIPUS treatment, LPS+0.5 W/cm² LIPUS group has a significant inhibitory effect in comparison to LPS group (average value±SEM; * represents comparison with the value of sham group, *P<0.05, **P<0.01, ***P<0.001; # represents comparison with the value of LPS group, #P<0.05, ##P<0.01, ###P<0.001; n=4).

The above results show the manifestation variation of TLR4 and NF-κB in cerebral cortex tissue after LIPUS abdomen treatment of the animal model of LPS-induced systemic inflammation. The LIPUS intensity 0.5 W/cm² has a significant effect on inhibiting TLR4 and NF-κB.

The present invention applies 0.5 W/cm² ultrasound to the abdomen. The cerebral cortex inflammation can be alleviated indirectly. The intensity of 1.0 W/cm² can effectively inhibit colonic inflammation and assist in improving the tight junction protein of colon. The tight junction protein of cerebral cortex is improved indirectly. The ultrasound at different intensities is applied to the abdomen. The biological effect range of abdominal ultrasound is not limited to the abdominal range, and influences the brain outside the range of ultrasonic irradiation. Therefore, the LIPUS is applicable to treating the neuroinflammation-related diseases induced via gut-brain axis.

The present invention relates to an ultrasound generator for inhibiting intestinal inflammatory factor and/or improving neuroinflammation. The system comprises a probe module, a processor module and an image module. A first probe of the probe module is connected to the ultrasound transducer, the first probe receives a first ultrasound signal of the ultrasound signals and transmits a first ultrasound outwards, furthermore, the ultrasound transducer receives a reflected ultrasound from a target area irradiated by the first ultrasound via the first probe, which is transmitted to the control unit. The image module is electrically communicated with the control unit, the control unit transmits the reflected ultrasound to the image module, the reflected ultrasound is converted by the image module and displayed on a display screen, the probe module is aligned with the target area by means of the display screen. A second probe of the probe module is connected to the ultrasound transducer, the second probe includes at least two independent piezo-patches, the control unit controls the ultrasound transducer to transfer a second ultrasound signal and a third ultrasound signal with a frequency difference to the piezo-patch. The second probe transmits a second ultrasound and a third ultrasound outwards from the second ultrasound signal and the third ultrasound signal through the piezo-patch, the second ultrasound and the third ultrasound focus in the target area, a beat frequency sound field (beating waves) is generated at the focus, the beating waves can achieve better inhibition of the tissue inflammation of the target area (intestinal tract) or/and indirect regulation of immune nervous and central nervous systems through the gut-brain axis.

An ultrasound generator probe for inhibiting intestinal inflammatory factor and/or improving neuroinflammation, the ultrasound generator probe comprises a first probe, the first probe is located in the center of the ultrasound generator probe with the purpose of imaging. a second probe is located in peripheral region of the device with the purpose of focusing probe. An ultrasound transducer, the ultrasound transducer is communicated with a first probe and a second probe and the second probe includes a plurality of piezo-patches. The ultrasound transducer controls the piezo-patches to send the ultrasounds with a plurality of frequency differences to a target area. The ultrasounds focus in the target area, a beat frequency sound field (beating waves) is generated in the focal area.

The present invention reduces specific neuron activity to recover the pathophysiologic manifestation of intestinal tract of mice (e.g. inflammatory reaction), promotes inflammatory disease associated therapeutic response and other immunity associated diseases.

### BRIEF DESCRIPTION OF THE DRAWINGS

FIG. 1 The block diagram of the ultrasound generator for inhibiting intestinal inflammatory factor and/or improving neuroinflammation.

FIG. 2 The probe module structure and beat frequency sound field.

FIG. 3 The structure of the first probe of probe module.

FIG. 4 The process of the ultrasound generator for inhibiting intestinal inflammatory factor and/or improving neuroinflammation.

FIG. 5a the 7 days' experiment data of mice.

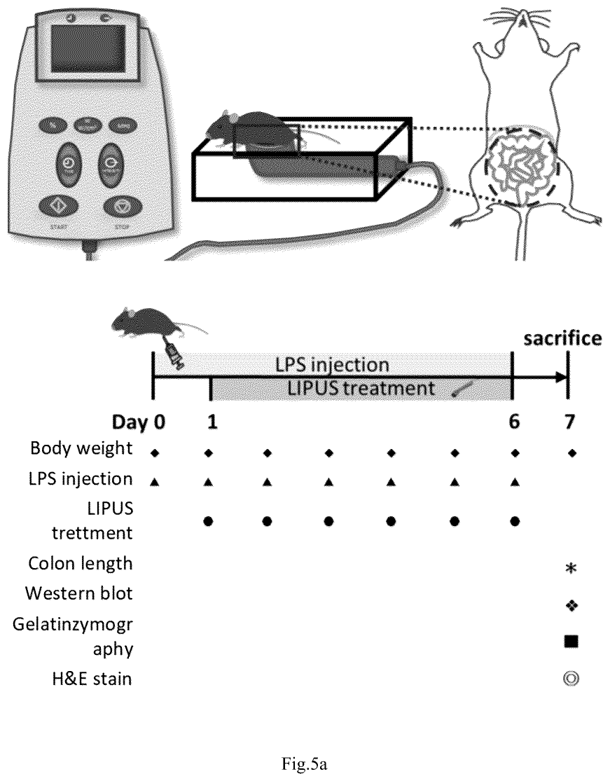

FIG. 5b the 7 days' experiment data of mice.

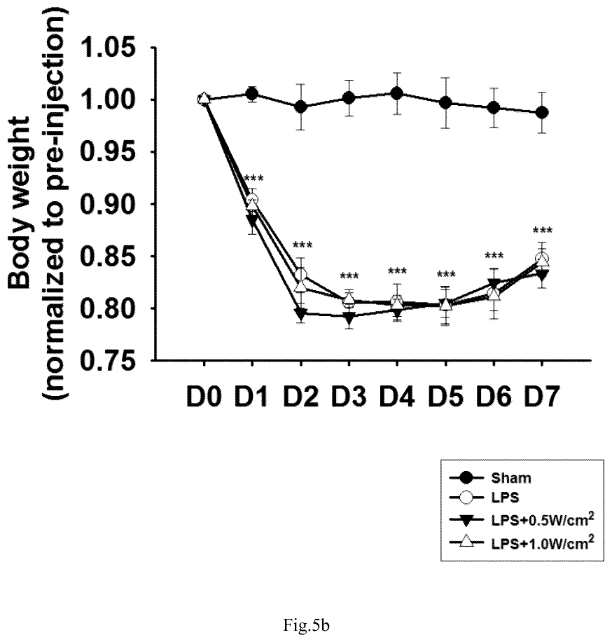

FIG. 6 The expression level of inflammatory factor and apoptosis factor in the colon tissue of laboratory mouse resulted from ultrasound stimulation in abdomen.

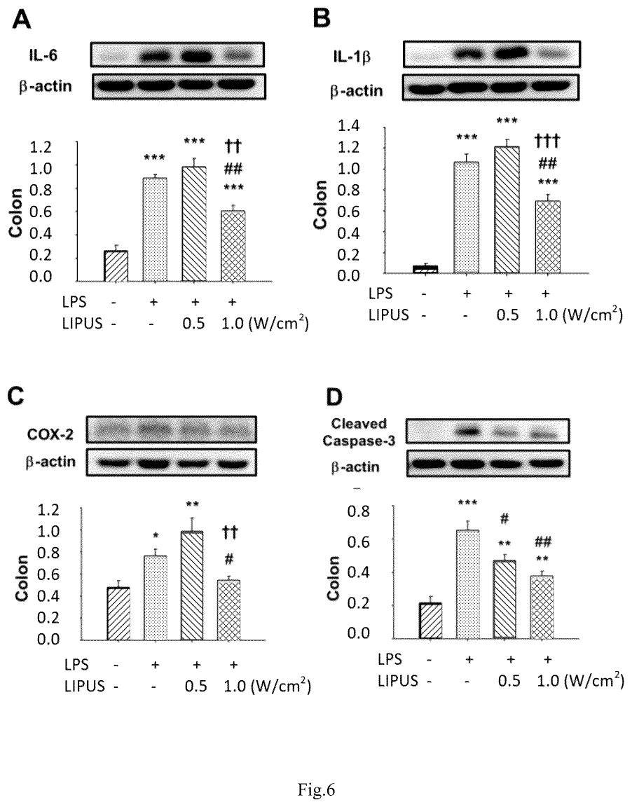

FIG. 7 The expression level of inflammatory factor and apoptosis factor in the cerebral cortex tissue of laboratory mouse resulted from ultrasound stimulation in abdomen.

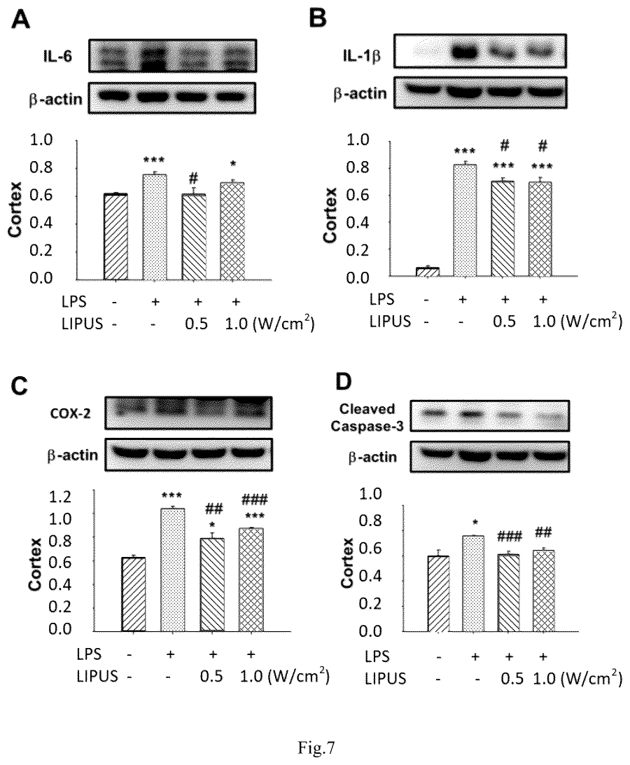

FIG. 8 The expression level of TLR4 receiver and NF-κB transcription factor in the cerebral cortex tissue of laboratory mouse resulted from ultrasound stimulation in abdomen.

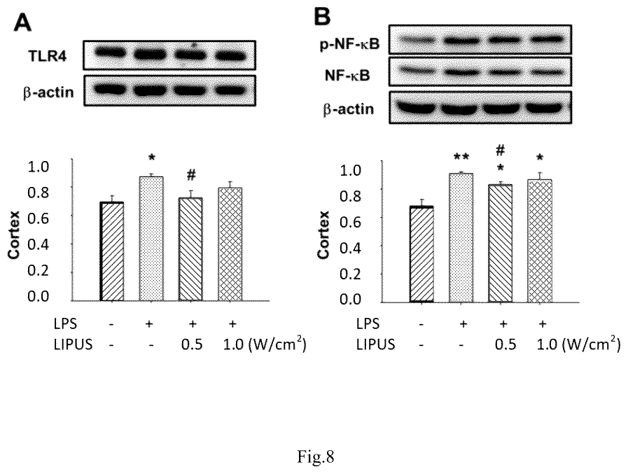

FIG. 9 The Influence of LIPUS on the Firmicutes-Bacteroidetes Ratio and Bacteroidetes-Proteobacteria Ratio in the colon.

### DETAILED DESCRIPTION OF THE INVENTION

### Embodiment 1

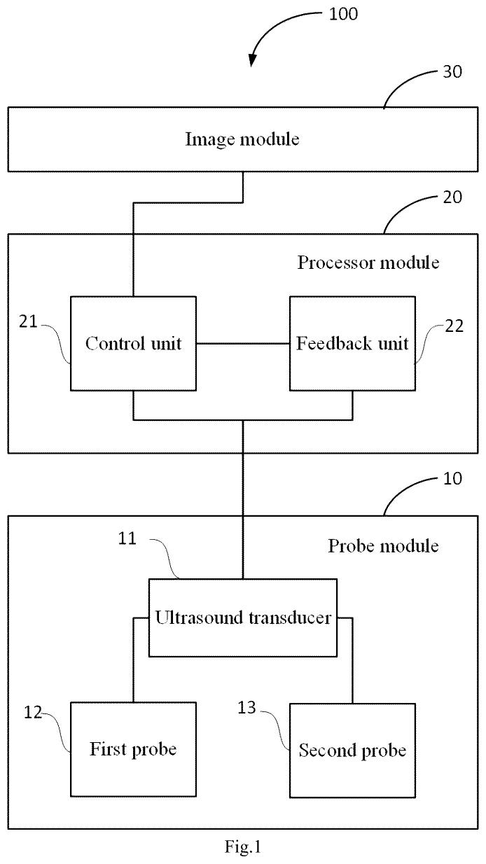

The present invention relates to an ultrasound generating system 100 which uses beat frequency sound field to inhibit intestinal inflammatory factor and/or to improve neuroinflammation. FIG. 1 is the block diagram of the ultrasound generator for inhibiting intestinal inflammatory factor and/or improving neuroinflammation. The system comprises a probe module 10, a processor module 20 and an image module 30.

The processor module 20 is electrically communicated with a power supply (not shown in the figure), a control unit 21 of the processor module 20 is electrically communicated with an ultrasound transducer 11 of the probe module 10, the control unit 21 regulates the ultrasound transducer 11 to send a plurality of ultrasound signals.

Ultrasonic transducer is a transducer that realizes mutual conversion of sound energy and electrical energy within the ultrasonic frequency range. It is mainly divided into three categories: transmitter, receiver, and transceiver. The transducer used to emit ultrasonic waves is called a transmitter. When the transducer is in the transmitting state, it converts electrical energy into mechanical energy and then into sound energy; the transducer used to receive sound waves is called a receiver. When the transducer is in the receiving state, it converts sound energy into mechanical energy and then into electrical energy; in some cases, the transducer can be used as both a transmitter and a receiver, which is called a dual-purpose transducer for sending and receiving. This technology belongs to the conventional technology commonly used in this field, and will not be repeated here.

A first probe 12 of the probe module 10 is connected to the ultrasound transducer 11, the first probe 12 receives a first ultrasound signal of the ultrasound signals and transmits a first ultrasound outwards, furthermore, the ultrasound transducer 11 receives a reflected ultrasound from a target area irradiated by the first ultrasound via the first probe 12, which is transmitted to the control unit 21.

The image module 30 is electrically communicated with the control unit 21, the control unit 21 transmits the reflected ultrasound to the image module 30, the reflected ultrasound is converted by the image module 30 and displayed on a display screen (not shown in the figure), the probe module 10 is aligned with the target area by means of the display screen.

A second probe 13 of the probe module 10 is connected to the ultrasound transducer 11, the second probe 13 includes at least two independent piezo-patches 131, the control unit 21 controls the ultrasound transducer 11 to transfer a second ultrasound signal and a third ultrasound signal with a frequency difference to the piezo-patch 131.

The second probe 13 transmits a second ultrasound and a third ultrasound outwards from the second ultrasound signal and the third ultrasound signal through the piezo-patch 131, the second ultrasound and the third ultrasound focus in the target area, so as to inhibit the tissue inflammation of the target area (intestinal tract) or/and to indirectly regulate immune nervous and central nervous systems via the gut-brain axis.

Preferably, a beat frequency sound field (beating waves) is generated at the focus, the beating waves can achieve better inhibition of the tissue inflammation of the target area (intestinal tract) or/and indirect regulation of immune nervous and central nervous systems through the gut-brain axis.

A feedback unit 22 of the processor module 20 detects a detection data of the ultrasounds transferred by the second probe 13, and feeds back the detection data to the control unit 21, the control unit 21 adjusts the ultrasound signals exported from the ultrasound transducer 11 according to the detection data, so as to stabilize the ultrasounds transferred by the second probe 13.

The processor module 20 includes a warning function, when the feedback unit 22 detects an abnormal phenomenon in the ultrasounds transferred by the second probe 13, the warning function gives a warning, the abnormal phenomena include offset settings, overheating and irradiation interruption.

The ultrasound transducer 11 generates the probe of sound field, the first probe 12 of the probe module 10 is a probe for localization (or diagnostic probe), for looking for the region to be irradiated by the second ultrasound and the third ultrasound, and assisting in localizing the second probe 13 in the irradiation process.

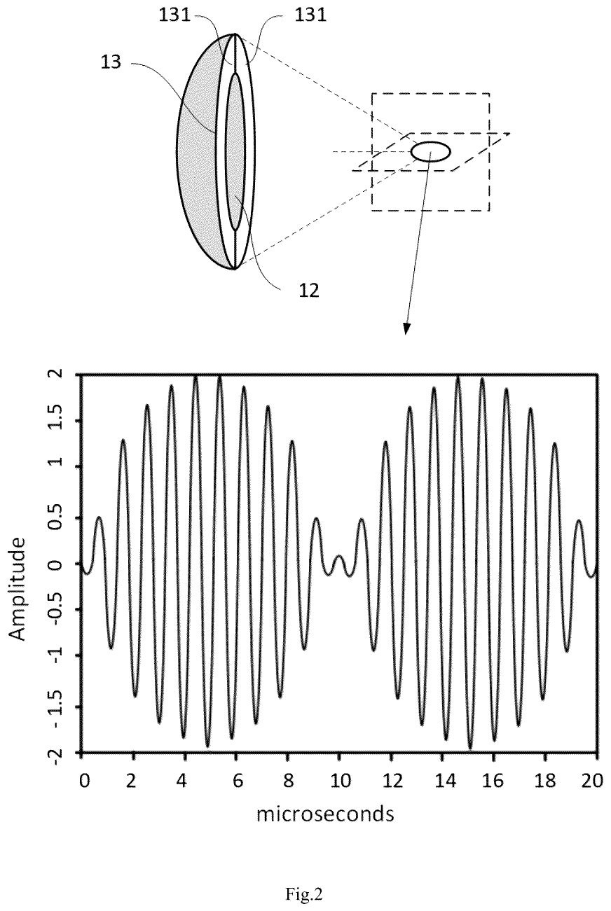

FIG. 2 shows the probe module structure and beat frequency sound field, the second probe 13 of the probe module 10 is a focusing probe, which can focus the second ultrasound and the third ultrasound with a frequency difference at a fixed range in front of the first probe 12 through the piezo-patches 131, the physical phenomenon of the beating waves is generated at the focus.

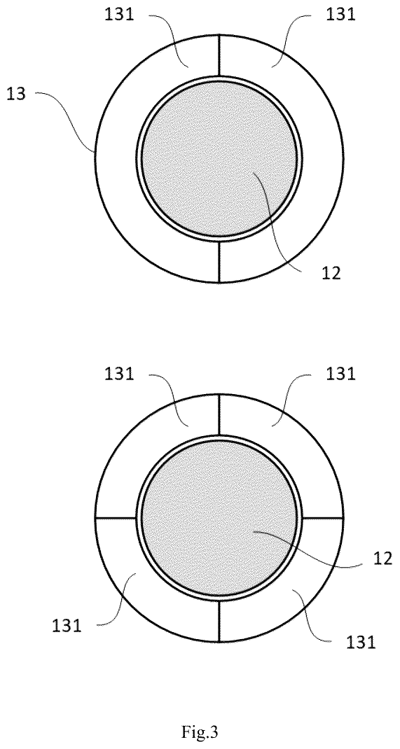

FIG. 3 shows the structure of the first probe of probe module, the first probe 12 and the second probe 13 of the probe module 10 are an integrated single probe, the first probe 12 is in the center of the integrated single probe, the second probe 13 is in the peripheral region of the integrated single probe.

An embodiment, as shown in FIG. 3, the second probe 13 of the probe module 10 includes a plurality of independent piezo-patches 131, the control unit 21 controls the ultrasound transducer 11 to transfer the ultrasound signals with the frequency difference, the ultrasound signals correspond to the piezo-patches 131.

The ultrasound generating system 100 which uses beat frequency sound field to inhibit intestinal inflammatory factor and/or to improve neuroinflammation of the present invention includes a wearable device, the wearable device can be equipped with a plurality of probe modules 10, the wearable device can be fixed to the abdomen, so that the probe modules 10 are aligned with specific positions.

The wearable device is fixed to the abdomen by using a girdle or/and Velcro, the irradiated position can be adjusted elastically or one probe or multiple probes can be fixed to one specific position or different positions for synchronous irradiation.

The first probe 12 is a diagnostic probe of the existing technology, with the purpose of imaging, this technology is a common ultrasound imaging technique of this domain, not to be described anymore.

An embodiment, the sound field frequency of the ultrasounds generated by the second probe 13 is 20 kHz˜20 MHz.

An embodiment, the frequency difference of the ultrasounds generated by the second probe 13 is less than or equal to 100 kHz.

An embodiment, the frequency difference of the ultrasounds generated by the second probe 13 is 10 kHz.

An embodiment, the sound power of the ultrasounds generated by the second probe 13 is 1 mW/cm²˜10 W/cm².

The second probe 13 outside the probe module 10 generates time varying sound field strength, the intestinal tract tissue inflammation can be inhibited or/and immune neuromodulation can be achieved through the gut-brain axis.

An embodiment, the control unit 21 includes a time setting function, the time setting function sets up the irradiation time of the ultrasounds transferred by the second probe 13, and sets up the irradiation time of the probe modules 10 respectively.

An embodiment, the image module 30 includes a localization setting, when the first probe 12 receives the reflected ultrasound from the target area, which is displayed on the display screen to complete localization, the image module 30 can record the target area image, if the target area image received by the first probe 12 offsets in the irradiation process, the warning function gives a warning.

The display screen displays an irradiated position marker to assist in localizing the target area.

The focusing distance of the ultrasounds transmitted from the piezo-patches 131 of the second probe 13 is fixed, an auxiliary probe can be arranged in front of the probe module 10 to change the focusing distance.

The focusing distance of the ultrasounds transmitted from the piezo-patches 131 of the second probe 13 is adjustable, the second probe 13 has an angle rotation function, the angle of the piezo-patches 131 can be changed, so as to change the focusing distance.

### Embodiment 2

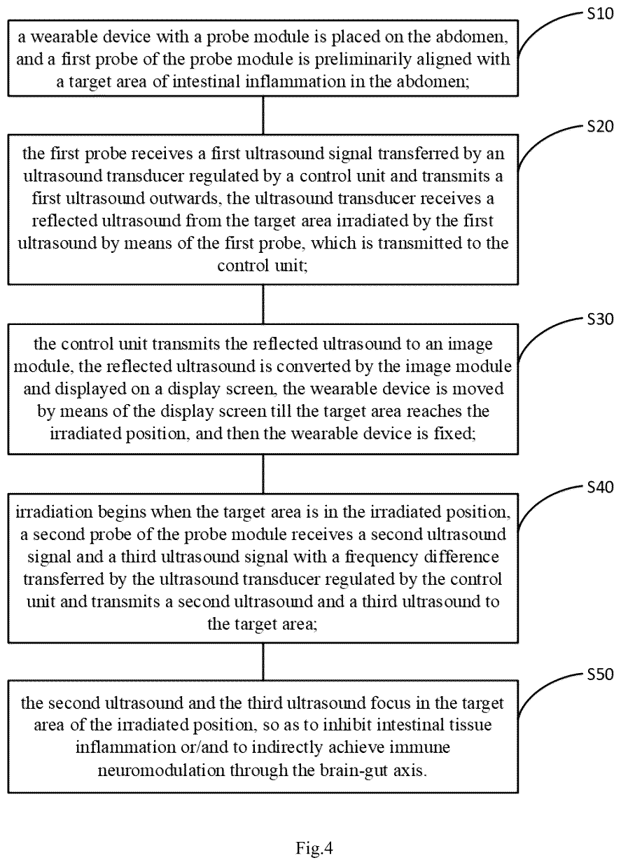

In terms of the process of the ultrasound generating system 100 which uses beat frequency sound field to inhibit intestinal inflammatory factor and/or to improve neuroinflammation for intestinal inflammation or/and immune neuromodulation of the present invention, FIG. 4 shows the process of the ultrasound generator for inhibiting intestinal inflammatory factor and/or improving neuroinflammation, the process includes S10. a wearable device with a probe module 10 is placed on the abdomen, and a first probe 12 of the probe module 10 is preliminarily aligned with a target area of intestinal inflammation in the abdomen; S20. the first probe 12 receives a first ultrasound signal transferred by an ultrasound transducer 11 regulated by a control unit 21 and transmits a first ultrasound outwards, the ultrasound transducer 11 receives a reflected ultrasound from the target area irradiated by the first ultrasound by means of the first probe 12, which is transmitted to the control unit 21; S30. the control unit 21 transmits the reflected ultrasound to an image module 30, the reflected ultrasound is converted by the image module 30 and displayed on a display screen, the wearable device is moved by means of the display screen till the target area reaches the irradiated position, and then the wearable device is fixed; S40. irradiation begins when the target area is in the irradiated position, a second probe 13 of the probe module 10 receives a second ultrasound signal and a third ultrasound signal with a frequency difference transferred by the ultrasound transducer 11 regulated by the control unit 21 and transmits a second ultrasound and a third ultrasound to the target area; S50. the second ultrasound and the third ultrasound focus in the target area of the irradiated position, so as to inhibit intestinal tissue inflammation or/and to indirectly achieve immune neuromodulation through the gut-brain axis.

The second ultrasound and the third ultrasound generate a beat frequency sound field (beating waves) at the focus, a better effect on inhibiting intestinal tissue inflammation or/and indirectly achieving immune neuromodulation through the gut-brain axis can be achieved by means of the beat frequency sound field.

A feedback unit 22 of the processor module 20 captures a detection data of the ultrasounds transferred by the second probe 13 in the irradiation process, the control unit 21 adjusts the output of the ultrasound transducer 11 according to the detection data, the ultrasound signals are stabilized.

When the detection data detected by the processor module 20 in the irradiation process is abnormal, this device gives a warning.

When the image module 30 detects an offset of the target area image displayed on the display screen in the irradiation process, this device gives a warning.

### Embodiment 3

In the embodiment of integrated ultrasound transducer and ultrasound probe, the ultrasound generating system 100 which uses beat frequency sound field to inhibit intestinal inflammatory factor and/or to improve neuroinflammation of the present invention. The system comprises at least a probe module 10, a processor module 20 and an image module 30; a control unit 21 of the processor module 20 is electrically communicated with one end of the probe module 10, the control unit 21 controls the probe module 10 to send a plurality of ultrasounds outwards from a plurality of probes at the other end; wherein a first probe 12 of the probes sends a first ultrasound to a target area, and then the reflected wave of the first ultrasound is received by the first probe 12, and converted into an image signal and sent to the control unit 21; the image module 30 is electrically communicated with the control unit 21, the image module 30 receives the image signal and displays the target area on a display screen; a second probe 13 of the probes includes at least two piezo-patches 131, the control unit 21 controls the piezo-patches 131 to send a second ultrasound and a third ultrasound with a frequency difference to the target area; and the second ultrasound and the third ultrasound focus in the target area, a beat frequency sound field (beating waves) is generated in the focal area.

### Embodiment 4

An ultrasound generator for inhibiting intestinal inflammatory factor and/or improving neuroinflammation, the device comprises an ultrasound transducer 11, the ultrasound transducer is electrically communicated with a first probe 12 and a second probe 13; the first probe 12 is located in the center of the device, one end of the first probe 12 is electrically communicated with the ultrasound transducer 11, the other end sends a first ultrasound to a target area; the second probe 13 is located in peripheral region of the device, surrounding the first probe 12, one end of the second probe 13 is electrically communicated with the ultrasound transducer 11, the other end sends a plurality of ultrasounds to the target area; and the second probe 13 includes a plurality of piezo-patches 131, the ultrasound transducer 11 controls the piezo-patches 131 to send the ultrasounds with a plurality of frequency differences to a target area.

The plurality of ultrasounds focus in the target area, a beat frequency sound field (beating waves) is generated in the focal area.

### Embodiment 5

A method of ultrasound generating system for inhibiting intestinal inflammatory factor and/or improving neuroinflammation, wherein a first probe 12 in the center of a probe module 10 is aligned with a target area of intestinal inflammation in the abdomen; a control unit 21 controls the first probe 12 to transmit a first ultrasound to the target area, a reflected ultrasound from the target area irradiated by the first ultrasound received and sent to an image module 30; the image module 30 receives the reflected ultrasound and displays the target area on a display screen; the control unit 21 controls a second probe 13 outside the probe module 10 to transmit a second ultrasound and a third ultrasound with a frequency difference to the target area; the second ultrasound and the third ultrasound are focused in the target area and generated a beat frequency sound field (beating waves) at the focus.

The better effect on inhibiting intestinal tissue inflammation or/and indirectly achieving immune neuromodulation through the gut-brain axis can be achieved by means of the beat frequency sound field.

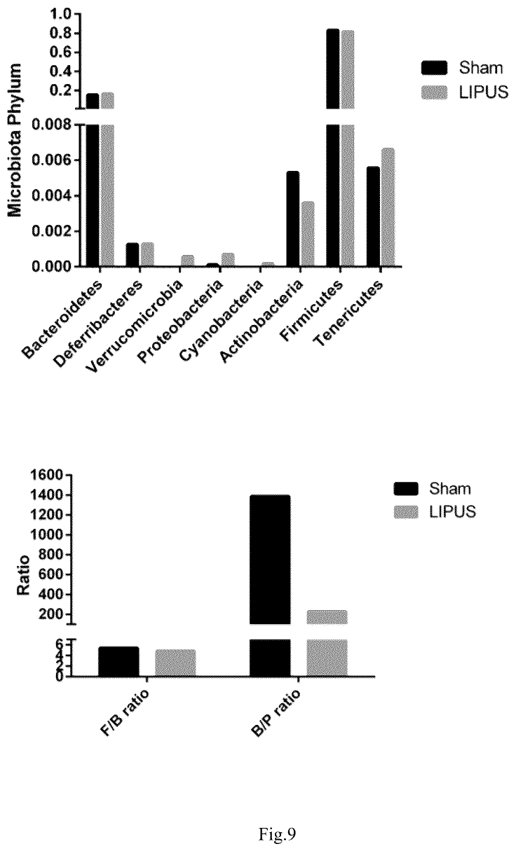

An embodiment, as shown in FIG. 9, the Influence of LIPUS generate a beat frequency sound field (beating waves) on the Firmicutes-Bacteroidetes Ratio and Bacteroidetes-Proteobacteria Ratio in the colon, the ratios between major bacterial phyla had been regarded as the significant relevance in composition, ecological alteration and the indication of dysbiosis of gut microbiota, whereas PS128 gavage (the same as LIPUS) resulted in a decline in F/B and B/P ratios, at the phylum level, the PS128 (the same as LIPUS) treated group showed a significant elevation in the Proteobacteria population, Psychiatric Disease: FIB ratio rise [symbol] IBD: FIB ratio decline.

Wherein the beating waves can modulate the expression level of inflammatory factor and apoptosis factor by inhibiting the mitigation mediated the pro-inflammatory pathway by TLR4/NF-κB.

Wherein the beating waves can modulate the colonic inflammatory factor and apoptosis factor, wherein the beating waves can inhibit the expression level of tight junction protein of cerebral cortex.

Wherein the gut microbial homeostasis can be regulated by the beating waves.

Wherein the beating waves can elevate Proteobacteria population, Verrucomicrobacteria population, Cyanobacteria population, or Tenericutes population in the colon of a subject.

Wherein the Proteobacteria population can elevate 548.61%, P((LIPUS-sham)/sham)*100∘

Wherein the beating waves can decrease Firmicutes-Bacteroidetes (F/B) ratio of 9.44%, F/B ratio ((LIPUS-sham)/sham)*100∘

Wherein the beating waves can decrease Bacteroidetes-Proteobacteria (B/P) Ratio 83.25%, B/P ratio ((LIPUS-sham)/sham)*100∘

Wherein the beating waves can inhibit chronic inflammation of tissues in the subject's digestive tract.

Wherein the subject selected from cat, dog, rabbit, cattle, horse, sheep, goat, monkey, mouse, rat, gerbil, guinea pig, pig and the human.

Wherein he beating waves can decrease incidence rate of inflammatory bowel disease (IBD).

Wherein the beating waves can modulate the immune neuromodulation through the gut-brain axis.

Wherein the beating waves can decrease incidence rate of psychiatric disease.

The gut-brain axis, also known as the gut-brain axis, is a communication bridge between the brain and the intestinal digestive tract, and the bacteria in the gut also contribute a lot to this path. The three Interacting and regulating various physiological functions of the whole body, from the early development of the brain to the neurological diseases of the late old age, are closely related to this connection axis. The gut-brain axis includes the central nervous system, the central endocrine system, and the central immune system, including the hypothalamus-pituitary-adrenal axis (HPA axis), the sympathetic nervous system in the autonomic nervous system, and the parasympathetic nervous system. The nervous system (vagus nerve) and enteric nervous system, as well as the microbiota in the gut are mainly connected and dominated by the autonomic nervous system on the gastrointestinal surface of animals, but under the stimulation of the bacteria in the gastrointestinal tract, they also It will promote intestinal epidermal cells to secrete physiological regulation information, and this physiological regulation information will not only induce local immune response, but also transmit physiological information to the brain center through the autonomic nervous system connected with it, and then affect the central endocrine system and central immune system.

Inflammation, also known as inflammation, inflammatory response, and inflammatory response, is a protective response involving immune cells, blood vessels, and molecular mediators. part of a complex biological response.

1. An ultrasound system for inhibiting intestinal inflammatory factor and/or improving neuroinflammation, the system comprises at least a probe module, a processor module and an image module; wherein a first probe of the probes sends a first ultrasound to a target area, and then the reflected wave of the first ultrasound is received by the first probe, and converted into an image signal and sent to a control unit; the image module is electrically communicated with the control unit, the image module receives the image signal and displays the target area on a display screen; a second probe of the probes includes at least two piezo-patches, the control unit controls the piezo-patches to send a second ultrasound and a third ultrasound with a frequency difference to the target area; and the second ultrasound and the third ultrasound focus in the target area.

2. The system defined in claim 1, the second ultrasound and the third ultrasound generate a beating waves at the target area.

3. The system defined in claim 1, the first probe and the second probe of the probe module are an integrated single probe, the first probe is in the center of the integrated single probe, the second probe is in the peripheral region of the integrated single probe.

4. The system defined in claim 1, the first probe is imaging probe and a second probe is focusing probe.

5. The system defined in claim 1, the system includes a wearable device, the wearable device can be equipped with a plurality of probe modules.

6. The system defined in claim 1, the sound field frequency of the ultrasounds generated by the second probe is 20 kHz˜20 MHz, the sound power is 1 mW/cm²˜10 W/cm², the frequency difference of the ultrasounds generated is less than or equal to 100 kHz.

7. The system defined in claim 1, the second probe includes a plurality of piezo-patches, the piezo-patches to send the ultrasounds with a plurality of frequency differences to a target area.

8. An ultrasound generator for inhibiting intestinal inflammatory factor and/or improving neuroinflammation, the device comprises an ultrasound transducer, the ultrasound transducer is electrically communicated with a first probe and a second probe; the first probe is located in the center of the device, one end of the first probe is electrically communicated with the ultrasound transducer, the other end sends a first ultrasound to a target area; the second probe is located in peripheral region of the device, surrounding the first probe, one end of the second probe is electrically communicated with the ultrasound transducer, the other end sends a plurality of ultrasounds to the target area; and the second probe includes a plurality of piezo-patches, the ultrasound transducer controls the piezo-patches to send the ultrasounds with a plurality of frequency differences to the target area.

9. The generator defined in claim 8, the plurality of ultrasounds focus in the target area, a beating waves is generated in the focal area.

10. The generator defined in claim 8, the sound field frequency of the ultrasounds generated by the second probe is 20 kHz˜20 MHz, the sound power is 1 mW/cm²˜10 W/cm², the frequency difference of the ultrasounds generated is less than or equal to 100 kHz.

11. An ultrasound method for inhibiting intestinal inflammatory factor and/or improving neuroinflammation, wherein a first probe in the center of a probe module is aligned with a target area of intestinal inflammation in the abdomen; a control unit controls the first probe to transmit a first ultrasound to the target area, a reflected ultrasound from the target area irradiated by the first ultrasound received and sent to an image module; the image module receives the reflected ultrasound and displays the target area on a display screen; the control unit controls a second probe outside the probe module to transmit a second ultrasound and a third ultrasound with a frequency difference to the target area; the second ultrasound and the third ultrasound are focused in the target area and generated a beating waves at the focus.

12. The method defined in claim 11, the inhibition of intestinal tissue inflammation or/and the immune neuromodulation through the gut-brain axis by the beating waves.

13. The method defined in claim 11, wherein the beating waves can modulate the expression level of inflammatory factor and apoptosis factor by inhibiting the mitigation mediated the pro-inflammatory pathway by TLR4/NF-κB.

14. The method defined in claim 11, wherein the beating waves can modulate the colonic inflammatory factor and apoptosis factor, wherein the beating waves can inhibit the expression level of tight junction protein of cerebral cortex.

15. The method defined in claim 11, wherein the gut microbial homeostasis can be regulated by the beating waves.

16. The method defined in claim 11, wherein the beating waves can elevate Proteobacteria population, Verrucomicrobacteria population, Cyanobacteria population, or Tenericutes population in the colon of a subject.

17. The method defined in claim 11, wherein the Proteobacteria population can elevate 548.61%.

18. The method defined in claim 16, wherein the beating waves can decrease Firmicutes-Bacteroidetes (F/B) ratio of 9.44%, or/and Bacteroidetes-Proteobacteria (B/P) Ratio 83.25%.

19. The method defined in claim 11, the sound field frequency of the ultrasounds generated by the second probe being 20 kHz˜20 MHz, the sound power being 1 mW/cm²˜10 W/cm², wherein the frequency difference of the ultrasounds generated is less than or equal to 100 kHz.
